(19) United States
(12) Patent Application Publication
(10) Pub. No.: US 2025/0127435 A1
(43) Pub. Date: Apr. 24, 2025

(54) BLOOD GLUCOSE SENSING SYSTEM

(71) Applicant: Masimo Corporation, Irvine, CA (US)

(72) Inventors: Kevin Hughes Pauley, Lake Forest, CA (US); Hoi Wong, Irvine, CA (US); Jesse Chen, Irvine, CA (US); Mathew Paul, Irvine, CA (US)

(21) Appl. No.: 18/954,270

(22) Filed: Nov. 20, 2024

Related U.S. Application Data
(63) Continuation of application No. 18/428,921, filed on Jan. 31, 2024, now Pat. No. 12,178,572.
Application No. 18/428,921 is a division of application No. 16/805,510, filed on Feb. 28, 2020, now abandoned.
Application No. 16/805,510 is a continuation of application No. 14/302,417, filed on Jun. 11, 2014, now abandoned.
(60) Provisional application No. 61/833,515, filed on Jun. 11, 2013.
(60) Provisional application No. 61/898,483, filed on Nov. 1, 2013.
(60) Provisional application No. 61/913,331, filed on Dec. 8, 2013.

(51) Int. Cl. A61B 5/145; A61B 5/00; G16H 50/20

(52) U.S. Cl. CPC A61B 5/14532; A61B 5/7275; G16H 50/20

(57) ABSTRACT

A blood glucose sensing system includes a plurality of physiological sensors. The system can estimate blood glucose based on discrete invasive blood glucose estimates from a blood sample, discrete noninvasive blood glucose estimates derived from optical sensors, and continuously-calculated blood glucose estimates derived from a nonlinear state-space model of glucose and insulin reactions within a human body. The state-space model has user-entered values corresponding to their insulin and meal intake. The user's blood glucose is estimated from a combination of the discrete invasive blood glucose estimates, the discrete noninvasive blood glucose estimates and the continuously-calculated blood glucose estimate.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND OF THE INVENTION

Noninvasive physiological monitoring systems for measuring constituents of circulating blood have advanced from basic pulse oximeters to monitors capable of measuring abnormal and total hemoglobin among other parameters. A basic pulse oximeter capable of measuring blood oxygen saturation typically includes an optical sensor, a monitor for processing sensor signals and displaying results and a cable electrically interconnecting the sensor and the monitor. A pulse oximetry sensor typically has a red wavelength light emitting diode (LED), an infrared (IR) wavelength LED and a photodiode detector. The LEDs and detector are attached to a patient tissue site, such as a finger. The cable transmits drive signals from the monitor to the LEDs, and the LEDs respond to the drive signals to transmit light into the tissue site. The detector generates a photoplethysmograph signal responsive to the emitted light after attenuation by pulsatile blood flow within the tissue site. The cable transmits the detector signal to the monitor, which processes the signal to provide a numerical readout of oxygen saturation (SpO₂) and pulse rate, along with an audible pulse indication of the person's pulse. The photoplethysmograph waveform may also be displayed.

Conventional pulse oximetry assumes that arterial blood is the only pulsatile blood flow in the measurement site. During patient motion, venous blood also moves, which causes errors in conventional pulse oximetry. Advanced pulse oximetry processes the venous blood signal so as to report true arterial oxygen saturation and pulse rate under conditions of patient movement. Advanced pulse oximetry also functions under conditions of low perfusion (small signal amplitude), intense ambient light (artificial or sunlight) and electrosurgical instrument interference, which are scenarios where conventional pulse oximetry tends to fail.

Advanced pulse oximetry is described in at least U.S. Pat. Nos. 6,770,028; 6,658,276; 6,157,850; 6,002,952; 5,769,785 and 5,758,644, all assigned to Masimo Corporation (“Masimo”) of Irvine, California and all hereby incorporated in their entireties by reference herein. Corresponding low noise optical sensors are disclosed in at least U.S. Pat. Nos. 6,985,764; 6,813,511; 6,792,300; 6,256,523; 6,088,607; 5,782,757 and 5,638,818, which are all also assigned to Masimo and are also all hereby incorporated in their entireties by reference herein. Advanced pulse oximetry systems including Masimo SET® low noise optical sensors and read through motion pulse oximetry monitors for measuring SpO₂, pulse rate (PR) and perfusion index (PI) are available from Masimo. Optical sensors include any of Masimo LNOP®, LNCS®, SofTouch™ and Blue™ adhesive or reusable sensors. Pulse oximetry monitors include any of Masimo Rad-8®, Rad-5®, Rad®-5v or SatShare® monitors.

Advanced blood parameter measurement systems are described in at least U.S. Pat. 7,647,083, filed Mar. 1, 2006, titled Multiple Wavelength Sensor Equalization; U.S. Pat. No. 7,729,733, filed Mar. 1, 2006, titled Configurable Physiological Measurement System; U.S. Pat. Pub. No. 2006/0211925, filed Mar. 1, 2006, titled Physiological Parameter Confidence Measure and U.S. Pat. Pub. No. 2006/0238358, filed Mar. 1, 2006, titled Noninvasive Multi-Parameter Patient Monitor, which are all assigned to Cercacor Laboratories, Inc., Irvine, CA (Cercacor) and all hereby incorporated in their entireties by reference herein. An advanced parameter measurement system that includes acoustic monitoring is described in U.S. Pat. Pub. No. 2010/0274099, filed Dec. 21, 2009, titled Acoustic Sensor Assembly, assigned to Masimo and herby incorporated in its entirety by reference herein.

Advanced blood parameter measurement systems include Masimo Rainbow® SET, which provides measurements in addition to SpO₂, such as total hemoglobin (SpHb™), oxygen content (SpOC™), methemoglobin (SpMet®), carboxyhemoglobin (SpCO®) and PVI®. Advanced blood parameter sensors include Masimo Rainbow® adhesive, ReSposable™ and reusable sensors. Advanced blood parameter monitors include Masimo Radical-7™, Rad-87™ and Rad-57™ monitors, all available from Masimo. Advanced parameter measurement systems may also include acoustic monitoring such as acoustic respiration rate (RRa™) using a Rainbow Acoustic Sensor™ and Rad-87™ monitor, available from Masimo. Such advanced pulse oximeters, low noise sensors and advanced parameter systems have gained rapid acceptance in a wide variety of medical applications, including surgical wards, intensive care and neonatal units, general wards, home care, physical training, and virtually all types of monitoring scenarios. Such advanced pulse oximeters, low noise sensors and advanced blood parameter systems have gained rapid acceptance in a wide variety of medical applications, including surgical wards, intensive care and neonatal units, general wards, home care, physical training, and virtually all types of monitoring scenarios.

### SUMMARY OF THE INVENTION

FIG. 1 generally illustrates a blood glucose measurement system 100 that advantageously combines relatively frequent noninvasive measurements of blood glucose interspersed with relatively infrequent invasive measurements of blood glucose so as to manage individual blood glucose levels. The blood glucose measurement system 100 has a blood glucose monitor 110, an optical sensor 120, a sensor cable 130 electrically and mechanically interconnecting the monitor 110 and sensor 120 and a monitor-integrated test strip reader that accepts test strips 150 via a test strip slot 140. In particular, the blood glucose measurement system 100 individually calibrates the noninvasive optical sensor 120 measurements with intermittent test strip measurements to provide the accuracy of individualized glucose test strip measurements at a much-reduced frequency of blood draws. Reduced blood draws are a substantial aid to persons who require frequent monitoring of blood glucose levels to manage diabetes and related diseases. In an embodiment, the monitor 110 has a handheld housing including an integrated touch screen 160 defining one or more input keys and providing a display of blood glucose levels among other features. An optical sensor is described in detail with respect to U.S. Pat. No. 13/646,659 titled Noninvasive Blood Analysis System, filed Oct. 5, 2012, assigned to Cercacor and hereby incorporated in its entirety by reference herein. A blood glucose monitor is described in detail with respect to U.S. Pat. No. 13/308,461 titled Handheld Processing Device Including Medical Applications for Minimally and Noninvasive Glucose Measurements, filed Nov. 30, 2011, assigned to Cercacor and hereby incorporated in its entirety by reference herein. A blood glucose monitor and sensor are described in U.S. Pat. No. 13/473,477 titled Personal Health Device, filed May 16, 2012, assigned to Cercacor and hereby incorporated in its entirety by reference herein.

FIGS. 2A-B illustrate a glucose monitor 200 having a optical sensor 210 input for generating noninvasive spot check estimates of blood glucose 252 and a test strip 220 input for generating invasive spot check estimates of blood glucose 252. As shown in FIG. 2A, a signal processor 230 analyzes the optical sensor 210 signals so as to generate the noninvasive spot check estimates. A strip reader 240 analyzes blood draw test strips 220 so as to generate the invasive spot check estimates. An output processor 250 integrates the noninvasive and invasive spot checks into a single blood glucose estimate [symbol] output 252. As shown in FIG. 2B, error measurements εᵢ, εₙ are incorporated into the blood glucose spot check measurements. The invasive 260 glucose measurement error is substantially less than the noninvasive 270 glucose measurement error εₙ.

One aspect of a blood glucose estimator has discrete invasive blood glucose values derived from a blood sample, discrete noninvasive blood glucose values derived from optical sensor data and modeled blood glucose values derived from a nonlinear state-space model of glucose and insulin reactions within a human body. The state-space model has user-entered values corresponding to insulin and meal intake. A glucose estimate is derived from a combination of the discrete invasive blood glucose values, the discrete noninvasive blood glucose values and the modeled blood glucose values.

In various embodiment, the modeled blood glucose values provide an interval of blood glucose values based upon simulation of extreme values of derivates of the state variables in the state-space model. The interval of blood glucose values collapses to an error εᵢ at the discrete invasive blood glucose values. The interval of blood glucose values collapses to an error εₙ at the discrete noninvasive blood glucose values. The parameters of the state-space model are dynamically optimized to minimize an error between calculated values of blood glucose and measured values of blood glucose. The values corresponding to insulin and meal intake are derived from weighted optical sensor data ratios. The weighted optical sensor data ratios are dynamically optimized to minimize an error between calculated values of blood glucose and measured values of blood glucose.

### BRIEF DESCRIPTION OF THE DRAWINGS

FIG. 1 is an illustration of a blood glucose measurement system;

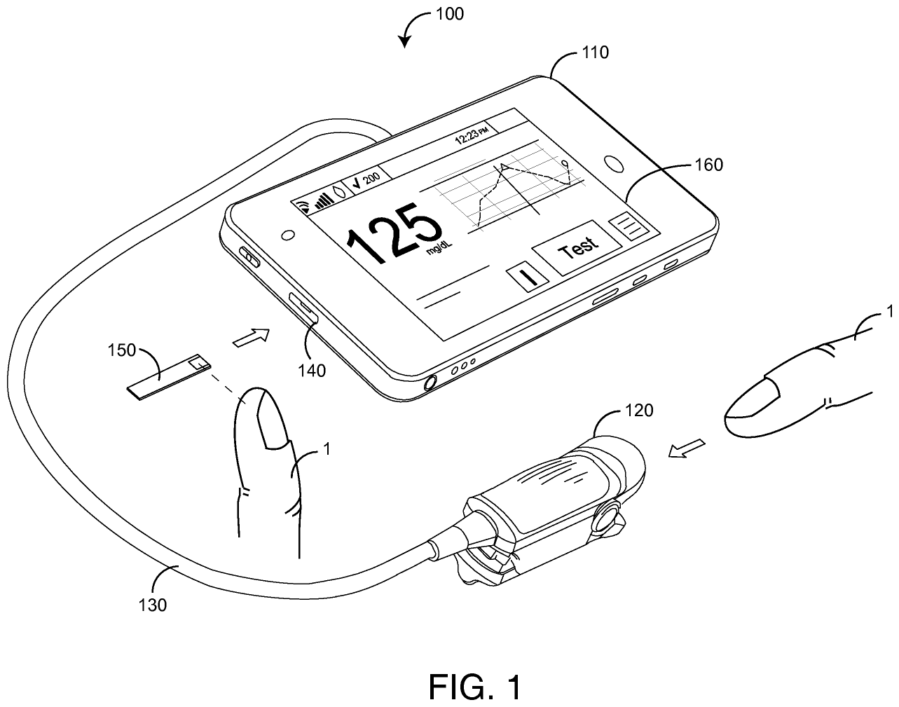

FIGS. 2A-B are a block diagram of a glucose monitor and a corresponding graph of discrete glucose estimates versus time;

FIGS. 3A-B are graphical display embodiments of a glucose trend and corresponding glucose trend intervals versus time;

FIG. 4 is a general block diagram of a blood glucose estimator;

FIG. 5 is a general block diagram of a nonlinear state-space model governing glucose and insulin reactions in the human body;

FIGS. 6A-B are a general block diagram of a discrete nonlinear state-space model governing glucose and insulin reactions in the human body and a graph of a corresponding glucose interval estimate;

FIGS. 7A-B are graphs of blood glucose interval estimates incorporating and responsive to both invasive and noninvasive (optical sensor) blood glucose spot checks;

FIG. 8 is a block diagram of dynamic optimization of a blood glucose-insulin model that inputs insulin and meal intake data and outputs modeled blood glucose values;

FIG. 9 is a block diagram of dynamic optimization of a blood glucose-insulin model having optical-sensor-generated light absorption ratio inputs for estimating insulin and meal intakes;

FIG. 10 is a detailed block diagram of a nonlinear state space model for glucose and insulin reactions in the human body; and

FIG. 11 is a blood glucose versus time graph comparing glucose model predictions to blood glucose measurements.

### DETAILED DESCRIPTION OF THE PREFERRED EMBODIMENT

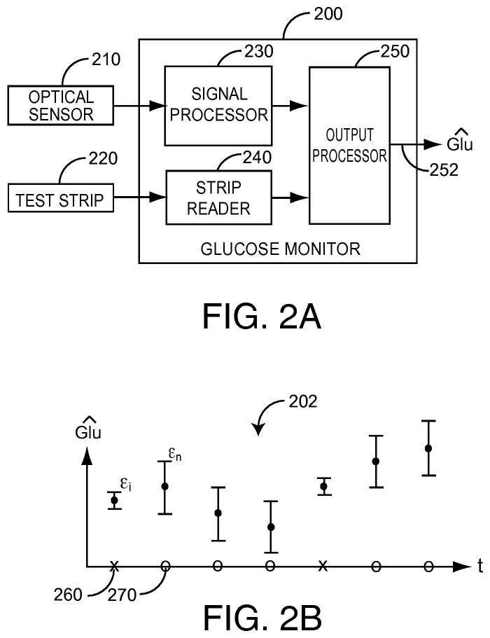

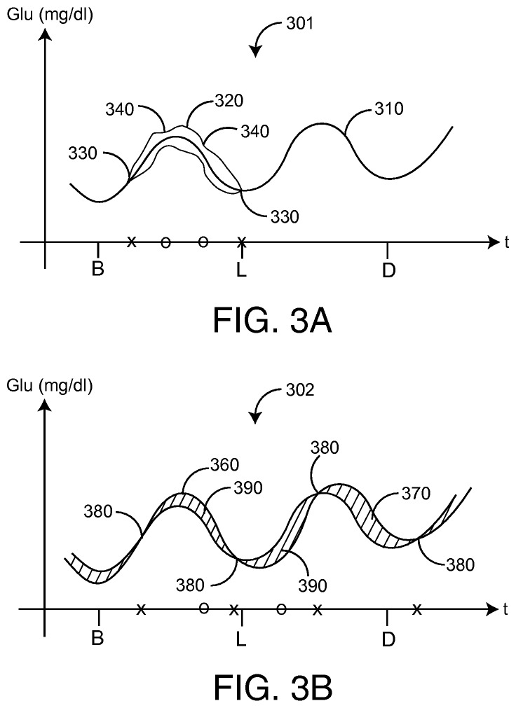

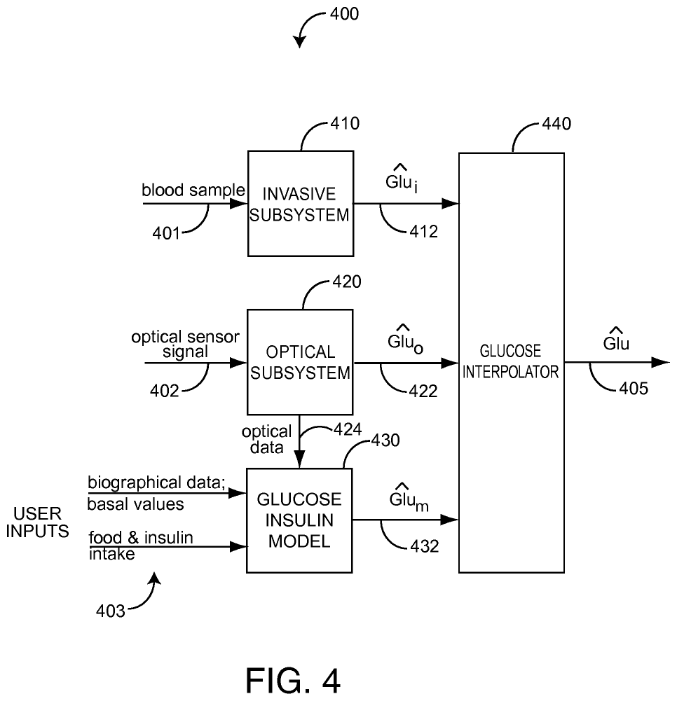

FIGS. 3A-B graphically illustrate interpolation between the discrete invasive (260 FIG. 2B) and noninvasive (270 FIG. 2B) measures described with respect to FIGS. 2A-B, above. FIG. 3A illustrates a glucose monitor output (252 FIG. 2A) embodiment 301 displaying a glucose trend 310 versus time. The trend 310 reflects, say, the rise and fall of glucose levels after breakfast (B), lunch (L) and dinner (D). An envelope 320 is generated to show trend accuracy. The envelope 320 generally coincides to the trend line 310 at invasive glucose spot checks (x) 330 and is reduced in breadth at noninvasive (optical) glucose spot checks (o) 340, reflecting the relative accuracy of these invasive and noninvasive measurements. FIG. 3B illustrates another glucose monitor output (252 FIG. 2A) embodiment 302 where the trend 360 has various areas 370, 380, 390 that reflect measurement accuracy. Invasive spot checks are reflected at trend nodes 380. Noninvasive spot checks are reflected at reduced size trend areas 390. Other areas 370 reflect an interpolation between glucose spot checks and modeled (calculated) glucose values so as to generate an estimated blood glucose output, as described with respect to FIG. 4, below. Interpolation between invasive and noninvasive glucose spot checks is described in detail with respect to interval simulation embodiments, below.

FIG. 4 generally illustrates a blood glucose estimator 400 having an invasive subsystem 410 for generating relatively accurate spot checks 412 of blood glucose utilizing a test strip and test strip reader (not shown). In particular, the test strip collects a blood sample 401 and a test strip reader reads the test strip to yield an invasive measure of blood glucose [symbol] 412. The glucose estimator 400 also has an optical subsystem 420 for generating less accurate, but painless, spot checks 422 of blood glucose using an optical sensor 402. In particular, the optical sensor 402 transmits light into a tissue site and detects the light after tissue attenuation to yield a noninvasive (optical) measure of blood glucose [symbol] 422. These spot checks 412, 422 provide discrete glucose inputs to a glucose interpolator 440, which generates a blood glucose estimate [symbol] 405 based upon [symbol] 412, [symbol] 422 and [symbol] 432, as described below.

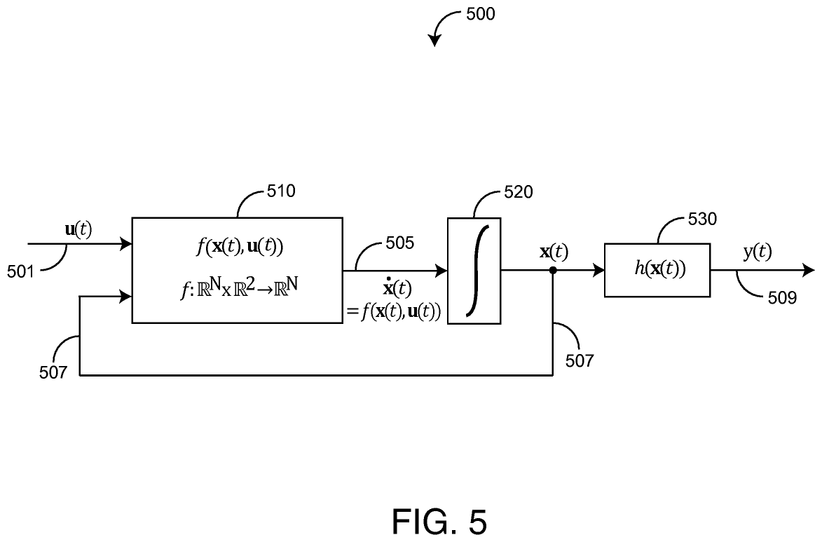

As shown in FIG. 4, a glucose insulin model 430 advantageously generates a continuous estimate of blood glucose [symbol] 432 over time between the glucose spot checks 412, 422. Further, the glucose insulin model 430 advantageously generates continuous glucose estimates versus time, as described in detail with respect to FIGS. 5-11, below. The glucose insulin model 430 inputs user provided data including biographical data, such as age and weight, basal values (user initial conditions) and food and insulin intake data 403. Advantageously, optical data 424 derived from the optical sensor 402 is also utilized by the glucose insulin model 430 so as to detect physiological events, such as food intake and insulin injections, independent of user inputs 403.

FIG. 5 generally illustrates a nonlinear state-space model 500 that provides a modeled glucose output y(t) based upon glucose and insulin reactions in the human body. The state-space model 500 advantageously predicts blood glucose levels 509 based upon a subject's food and insulin intake 501. In particular, the state-space model 500 has a state equation block 510 that outputs the mathematical description 505 of the glucose and insulin reactions in the human body; a state vector block 520 that solves that mathematical description to generate state variables 507 and an output block 530 that generates a modeled blood glucose 509 output.

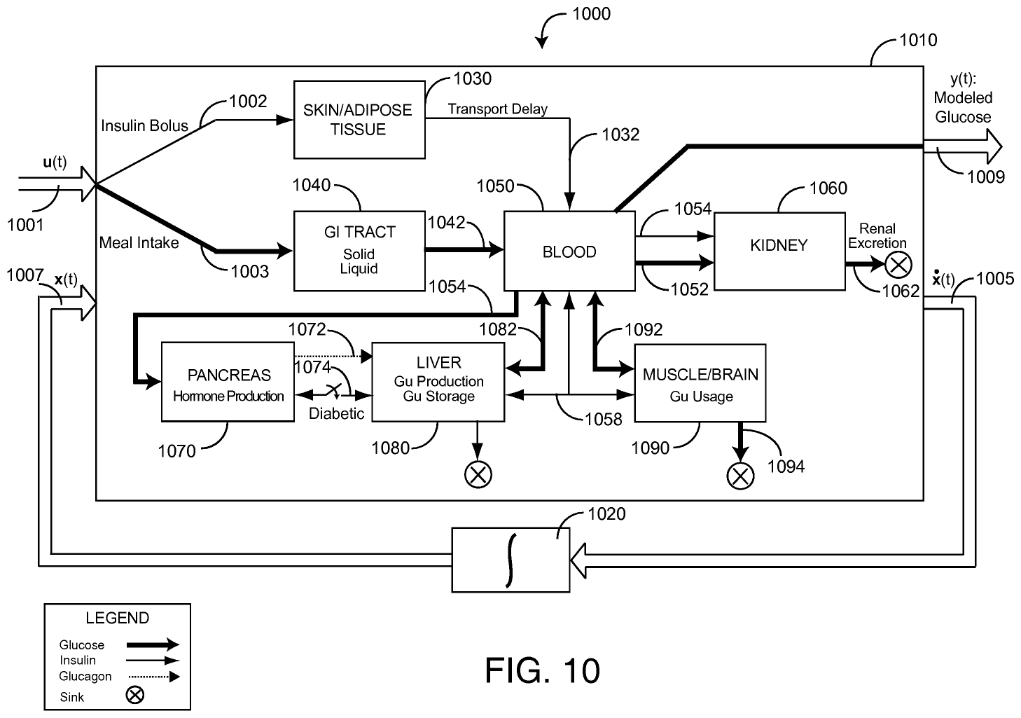

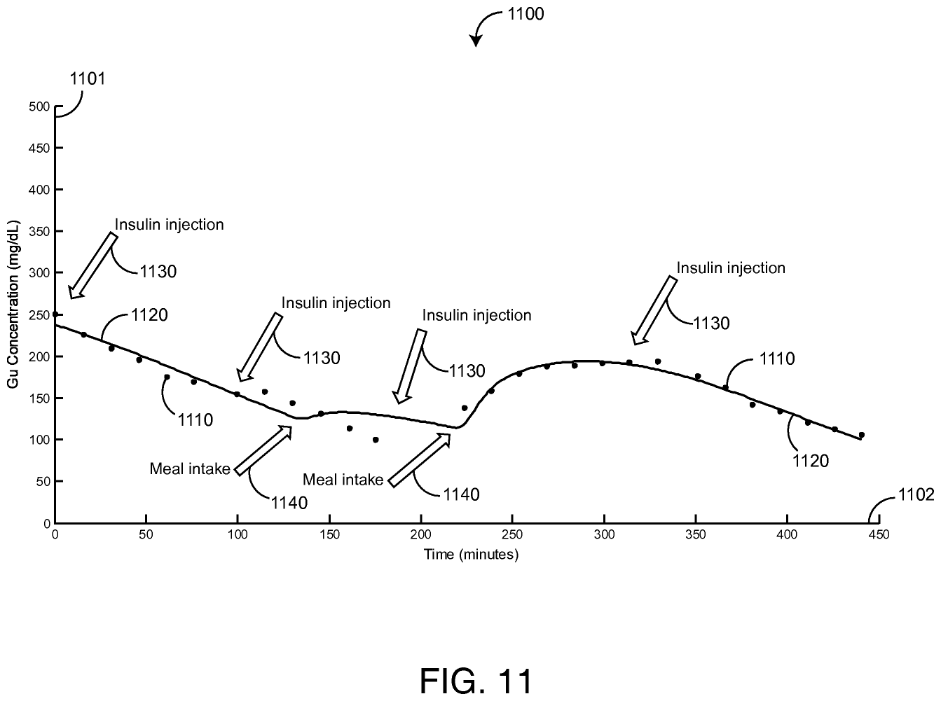

As shown in FIG. 5, in an embodiment, the state equation block 510 has an input vector u(t) 501 of insulin and food intake. The state equation block 510 generates an N-dimensional state equation \dot{x}(t) 505 from the input vector u(t) 501 and a state vector x(t) 507. The state vector block 520 solves the state equation \dot{x}(t) 505 to output the N-dimensional state vector x(t) 507. The output block 530 generates the modeled blood glucose y(t) 509 from the state vector x(t) 507. In an embodiment, the input vector u(t) 501 is a two-dimensional vector of insulin intake IIR(t) and glucose intake D(t) and the output y(t) 509 is a modeled blood glucose [symbol] , as described in detail with respect to FIGS. 10-11, below.

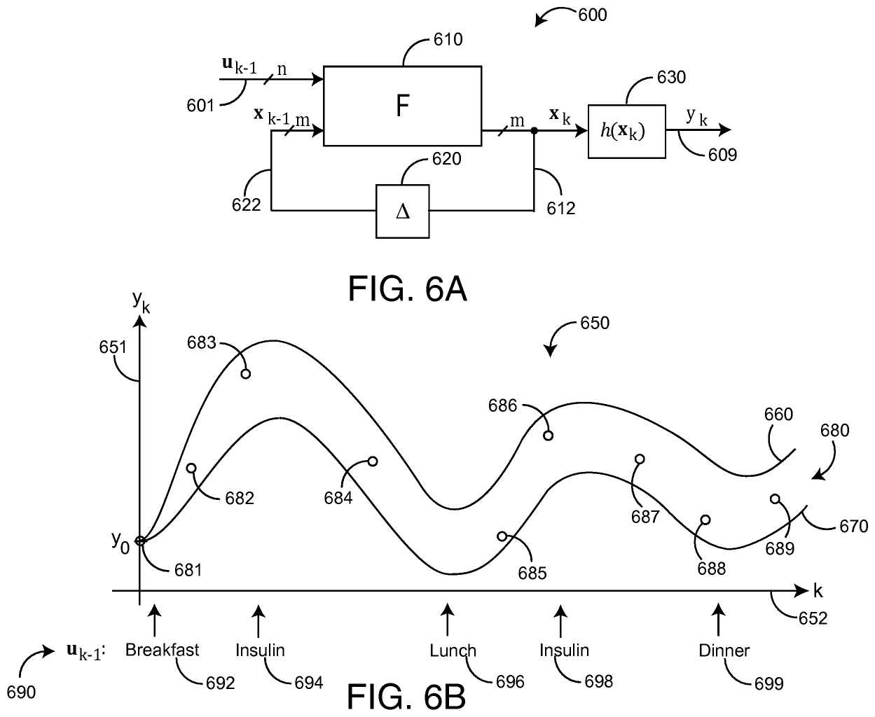

FIGS. 6A-B generally illustrate a discrete nonlinear state-space model 600 (FIG. 6A) and graphically illustrate corresponding interval estimates 650 (FIG. 6B) resulting from solutions to the state-space model differential equations. As shown in FIG. 6A, the discrete state-space model 600 has a state-space function block F 610 having an input vector uₖ₋₁ 601 and a state vector xₖ 612 output. The state vector xₖ 612 is delayed one discrete time interval 620 to generate a delayed state vector xₖ₋₁ 622 input to the function block F 610. An output block 630 inputs the state vector xₖ 612 and generates a modeled blood glucose level yₖ 609 output over discrete time k. State-space equations F 610 are a discrete time version of the state equation block 510 (FIG. 5) and state vector block 520 (FIG. 5) described above.

As shown in FIG. 6B, blood glucose interval estimates 650 derived from the state-space model 600 (FIG. 6A) are plotted on a blood glucose yₖ 651 axis versus a k (discrete time) axis 652. Numerical interval simulation (NIS) determines a glucose envelope 660, 670 for the glucose model state variable derivatives 612, which are described in further detail with respect to FIG. 10, below. In an embodiment, Runge-Kutta fourth order (RK4) NIS is used for calculating the envelope 660, 670.

The result are “fuzzy” blood glucose outputs yₖ 660, 670. The input vector uₖ₋₁ 601 (FIG. 6A) includes food and insulin intakes 690. Also shown for reference are blood glucose spot checks 680, which can be invasive (test strip) or noninvasive (optical sensor).

Further shown in FIG. 6B are exemplar spot checks 680 and intakes 690. For example, the intervals 660, 670 each start from an initial blood glucose spot check, y₀ 681. Later in the day, breakfast is eaten 692. Sometime later, another spot check 682 is taken, showing a rise in blood glucose. A further spot check 683 at a later time reveals a relatively high blood glucose, prompting an insulin intake 694. Blood glucose drops 660, 670, as verified by an additional spot check 684.

Continuing with respect to FIG. 6B, lunch is eaten 696. A later spot check 685 shows a blood glucose rise, followed by a further spot check 686 showing a still higher blood glucose and prompting another insulin intake 698. A couple later spot checks 687, 688 show that blood glucose is decreasing in response. Dinner is eaten 699. The latest spot check 689 shows glucose rising once again. In an embodiment, blood glucose interval estimates 660, 670 are combined with the spot checks 680. For example, calculations of the interval estimates 660, 670 begin at a new initial condition for each spot check 680. In an embodiment, the new initial conditions are at the spot check value for invasive spot checks and are at an interval for noninvasive (less accurate) spot checks.

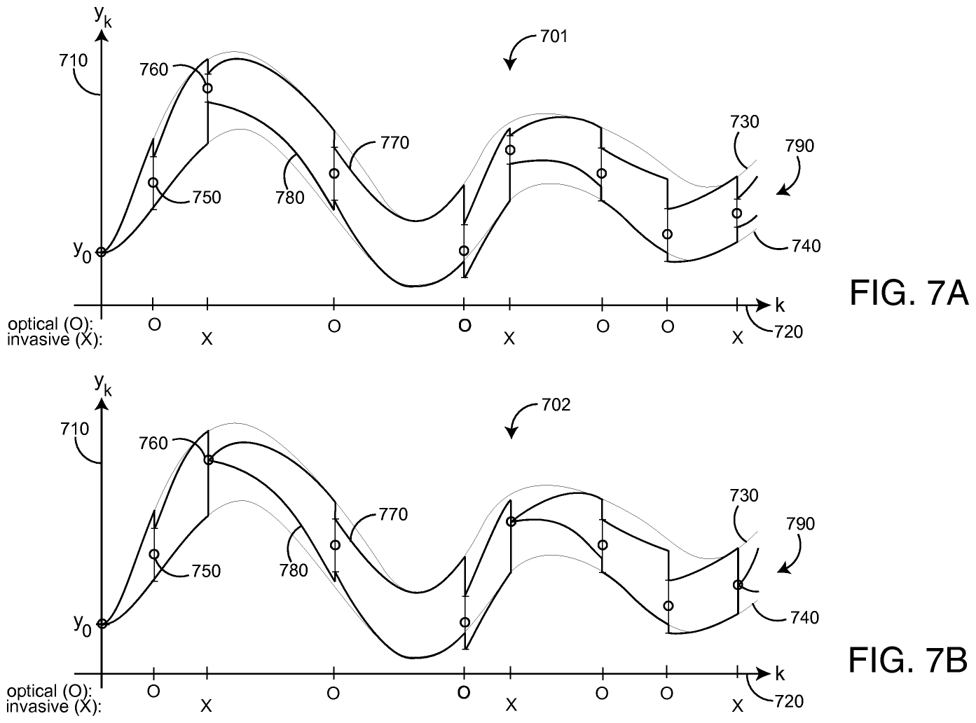

FIGS. 7A-B illustrate blood glucose interval estimates where the state-space model 600 (FIG. 6A) incorporates and is responsive to both invasive and noninvasive blood glucose spot checks. In an embodiment, blood glucose interval estimates 730, 740 are combined with the spot checks 790.

As shown in FIG. 7A, in an embodiment 701, calculations of the upper interval estimate 730 and lower interval estimate 740 begin at new (upper and lower) initial conditions for each spot check 790. In an embodiment, the interval for noninvasive (less accurate) spot checks 750 is larger than the interval for invasive (more accurate) spot checks 760. The result is a revised upper estimate 770 and revised lower estimate 780. In general, the interval between revised upper and lower estimates 770, 780 is less (more accurate) in view of the increased accuracy of the glucose-insulin model in view of the optical and invasive spot check measurements.

As shown in FIG. 7B, in an embodiment 702, the noninvasive spot checks 750 provide a spot check interval and the invasive spot checks 760 provide a spot check point. The result is a revised upper estimate 770 and revised lower estimate 780 for noninvasive spot checks 750 and a revised point estimate 760 for invasive spot checks.

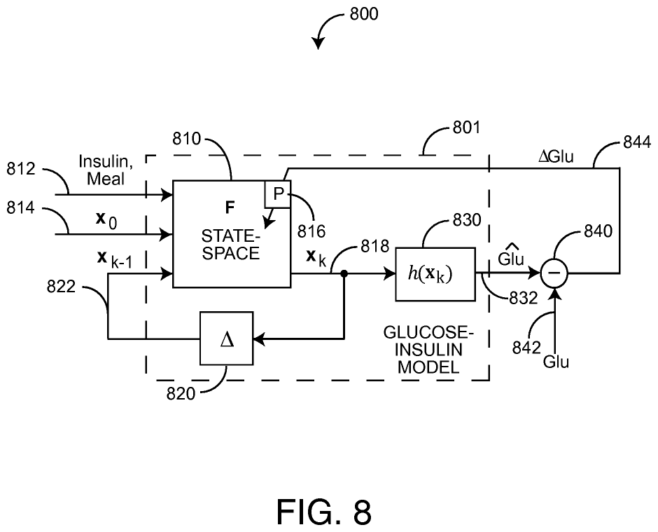

FIG. 8 generally illustrates a dynamic optimizer 800 for a glucose-insulin model 801. The glucose-insulin model 801 has a state-space function (F) block 810, a delay block 820 and an output function block 830, as described with respect to FIGS. 6A-B, above. The glucose-insulin model 801 has insulin and meal 812 and state variable initialization x₀ 814 inputs and generates an a blood glucose model [symbol] 832 output. The glucose-insulin model 801 is described generally with respect to FIGS. 4-5, above, and a glucose-insulin model embodiment is described in detail with respect to Appendix A, attached hereto.

As shown in FIG. 8, the state-space function block F 810 generates a xₖ 818 state variable output based upon a predetermined set of parameters P 816. State variable xₖ 818 is delayed to generate a delayed state variable xₖ₋₁ 822 input. The output function h(xₖ) block 830 generates the blood glucose model output [symbol] 832. Dynamic optimization repeatedly calculates a difference 840 of measured blood glucose Glu 842 and calculated blood glucose [symbol] 832 so as to generate ΔGlu 844. The parameters P 816 are recursively adjusted so as to minimize ΔGlu 844. The resulting optimized parameters are then locked into the state-space function block F 810.

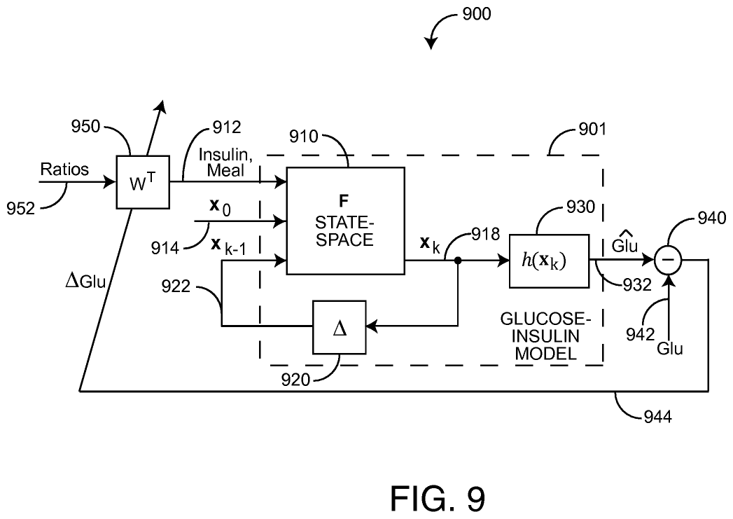

FIG. 9 generally illustrates an alternative dynamic optimizer 900 for a glucose-insulin model 901 that utilizes optical-sensor-derived light absorption ratios 952 in lieu of insulin and meal inputs 812 (FIG. 8). The glucose-insulin model 901 has a state-space functions (F) block 910, a delay block 920 and an output function block 930, as described with respect to FIG. 8, above. The glucose-insulin model 901 has estimated insulin and meal 912 inputs and state variable initialization x₀ 914 inputs and generates an a blood glucose model output [symbol] 932. The glucose-insulin model 901 is described generally with respect to FIG. 8, above, and a glucose-insulin model embodiment is described in detail with respect to FIG. 10, below.

As shown in FIG. 9, insulin and meal 912 inputs are estimated by a weighted sum of optic sensor-derived ratios 952, where the where the weights are dynamically optimized to minimize the difference ΔGlu 944 between the model-derived glucose output [symbol] 932 and measured blood glucose Glu 942. The resulting optimized weights 950 are then “locked-in” for estimating the insulin, meal inputs 912.

FIG. 10 illustrates a nonlinear state space model 1000 governing glucose and insulin reactions in the human body. The model advantageously predicts blood glucose levels 1009 based upon a subject's food and insulin intake 1001. In particular, the state space model 1000 has a state equation block 1010 that outputs the mathematical description 1005 of the glucose and insulin reactions in the human body; a state vector block 1020 that solves that mathematical description to generate state variables 1007 and an output block 1030 that generates a modeled blood glucose output 1009.

As shown in FIG. 10, in an embodiment, the state equation block 1010 has an input vector u(t) 1001 of insulin and food intake. The state equation block 1010 generates an N-dimensional state equation \dot{x}(t) 1005 from the input vector u(t) 1001 and a state vector x(t) 1007. The state vector block 1020 solves the state equation \dot{x}(t) 1005 to output the N-dimensional state vector x(t) 1007. The output block 1030 generates a modeled blood glucose y(t) output 1009 from the state vector x(t) 1007. In an embodiment, the input vector u(t) 1001 is a two-dimensional vector of insulin intake IIR(t) and glucose intake D(t) and the output y(t) 1009 is a blood glucose level G(t). Table I summarizes this glucose predictor model.

TABLE 1

| | |
|---|---|
| Inputs (u ∈ [symbol] ²) | Insulin intake IIR(t) and glucose intake D(t). |
| Parameters: | Patient body weight (kg), basal |
| | insulin I_{b} (pmol/L) if insulin is |
| | secreted and basal exogenous |
| | insulin infusion rate IIR_{b} |
| | (pmol/kg) if insulin is injected, |
| | basal blood glucose level G_{b} and basal |
| | endogenous glucose production EGP_{b}. |
| Output (y ∈ [symbol] ) | Blood glucose level G(t). |

Also shown in FIG. 10, the state space model 1000 has a state equation block 1010 and a state vector block 1020. An input u(t) 1001 to the state equation block 1010 describes insulin bolus 1002 and meal 1003 intakes and models various body functions. The state equation block 1010 models the response of various body organs and fluids to the insulin 1002 and meal 1003 intakes. The state vector block 1020 solves the state equation block 1010 to generate the state x(t) 1007, which is also input to the state equation block 1010. In response to the input u(t) and state x(t), the state equation block 1010 generates an output y(t) 1009 of modeled blood glucose.

As shown in FIG. 10, the physiological compartments in the model 1000 include skin and adipose (fat) tissue 1030, the GI tract 1040, blood 1050, kidneys 1060, the pancreas 1070, the liver 1080 and the brain and muscles 1090. A meal intake 1003 is digested in the GI tract 1040, which transports glucose 1042 to the blood stream 1050, which provides a modeled blood glucose y(t) 1009 output. The kidneys 1060 filter out some blood glucose 1052, which is excreted 1062. The pancreas 1070 converts some blood glucose 1054 to glucagon 1072, which is stored in the liver 1080. The liver 1080 also regulates blood glucose 1082 via glucose production to and storage from the blood stream 1050. The muscles and brain 1090 use a substantial quantity 1094 of blood glucose, exchanging blood glucose 1092 with the blood stream 1050 in the process.

Further shown in FIG. 10, insulin 1002 is injected into the skin/adipose tissue 1030, which enters the blood stream 1050 after a transport delay 1032. The kidneys 1060 filter out some insulin 1054, which is excreted 1062. The blood stream 1050 exchanges insulin 1058 with the liver 1080 and muscle/brain 1090. In a diabetic 1074, insulin 1074 is exchanged between the pancreas 1070 and liver 1080.

The state equation \dot{x}(t) is slightly different between types of subjects, i.e. those who are normal, those who have type I diabetes and those who have type II diabetes. In particular, \dot{x}(t) distinguishes subjects who secrete insulin and inject insulin, as shown in Table 2, below.

TABLE 2

| | Insulin Secreted | Insulin Injected |
|---|---|---|
| Normal | Yes | No |
| Type I | No | Yes |
| Type II | Yes | Yes |

EQS. 1-2 are the state variable x(t) and state equation \dot{x}(t) according to FIG. 10, described above. Table 3, below describes the individual elements of the state variable x(t).

$$\begin{matrix}x ( t ) & = & [ \begin{matrix}Y ( t ) \\ {G}_{plasma} ( t ) \\ {G}_{tissue} ( t ) \\ X ( t ) \\ {I}_{liver} ( t ) \\ {I}_{plasma} ( t ) \\ {I}_{portal} ( t ) \\ {I}_{1} ( t ) \\ {I}_{d} ( t ) \\ {Q}_{solid} ( t ) \\ {Q}_{liquid} ( t ) \\ {Q}_{gut} ( t ) \\ {I}_{poly 1} ( t ) \\ {I}_{mono 1} ( t ) \\ ⋮ \\ {I}_{ployN} ( t ) \\ {I}_{monoN} ( t ) \\ GL ( t ) \\ GLY ( t ) \\ {A}_{GL} ( t )\end{matrix} ] & EQ . 1 \\ & = & [ \begin{matrix}\dot{x} ( t ) \\ - α ( Y ( t ) - max ( - {S}_{b} , β ( \frac{{G}_{plasma} ( t )}{{V}_{G}} - h ) ) ) \\ EGP ( t ) - {GLY}_{r} + Ra ( t ) - {U}_{ii} ( t ) - E ( t ) - \\ {k}_{1} {G}_{plasma} ( t ) + {k}_{2} {G}_{tissue} ( t ) \\ - {U}_{id} ( t ) + {k}_{1} {G}_{plasma} ( t ) - {k}_{2} {G}_{tissue} ( t ) \\ - {p}_{2 U} X ( t ) + {p}_{2 U} ( \frac{{I}_{plasma} ( t )}{{V}_{1}} - {I}_{b} ) \\ - ( {m}_{1} + {m}_{3} ( t ) ) {I}_{liver} ( t ) + {m}_{2} {I}_{plasma} ( t ) + γ {I}_{portal} ( t ) \\ - ( {m}_{2} + {m}_{4} ) {I}_{plasma} ( t ) + {m}_{1} {I}_{liver} ( t ) + {R}_{i} ( t ) \\ - γ {I}_{portal} ( t ) + Y ( t ) + {S}_{b} + max ( 0 , K \frac{{G}_{plasma} ( t )}{{V}_{G}} ) \\ - {k}_{i} {I}_{1} ( t ) + \frac{{k}_{i}}{{V}_{I}} {I}_{plasma} ( t ) \\ {k}_{i} {I}_{1} ( t ) - {k}_{i} {I}_{d} ( t ) \\ - {k}_{grind} {Q}_{solid} ( t ) + D ( t ) \\ {k}_{grind} \cdot {Q}_{solid} ( t ) - {k}_{empty} ( t ) \cdot {Q}_{liquid} ( t ) \\ {k}_{empty} ( t ) \cdot {Q}_{liquid} ( t ) - {k}_{absorb} \cdot {Q}_{gut} ( t ) \\ - ( {k}_{d} + {k}_{a 1} ) {I}_{poly [ brand 1 ]} ( t ) + {IIR}_{brand 1} ( t ) \\ {k}_{d} {I}_{poly [ brand 1 ]} ( t ) - {k}_{a 2} {I}_{mono [ brand 1 ]} ( t ) \\ ⋮ \\ - ( {k}_{d} + {k}_{a 1} ) {I}_{poly [ brand N ]} ( t ) + {IIR}_{[ brand N ]} ( t ) ( t ) \\ {k}_{d} {I}_{poly [ brand N ]} ( t ) - {k}_{a 2} {I}_{mono [ brand N ]} ( t ) \\ - {k}_{GL} GL + {GL}_{b} ( \frac{{t}_{INS}}{{t}_{INS} + \frac{{I}_{L}}{{V}_{I}}} ) + {GL}_{b , r} ( \frac{1}{1 + {( \frac{G}{{t}_{G}} )}^{{η}_{G}}} ) * \\ \begin{matrix}( \frac{1}{1 + {( \frac{( GL ( t - {t}_{delay} )}{{t}_{GL}} )}^{{η}_{GL}}} ) * ( \frac{{t}_{INS}}{{t}_{INS} + \frac{{I}_{L}}{{V}_{I}}} ) \\ ( {GLY}_{r} - {GLY}_{d} ) body weight \\ {k}_{{GL}_{A}} ( \frac{tanhfun ( {GL}_{M} , \frac{GL}{{GL}_{b}} ) - 1}{2} - {A}_{GL} )\end{matrix}\end{matrix} ] & EQ . 2\end{matrix}$$

TABLE 3

| State Variable | Meaning | Dimensions |
|---|---|---|
| Y | Insulin Secretion Rate | pmol/(kg*min) |
| Gₚₗₐₛₘₐ | Plasma Glucose | mg/kg |
| Gₜᵢₛₛᵤₑ | Tissue Glucose | mg/kg |
| X | Insulin in interstitial fluid | pmol/L |
| Iₗᵢᵥₑᵣ | Insulin in liver | pmol/kg |
| Iₚₗₐₛₘₐ | Insulin in plasma | pmol/kg |
| Iₚₒᵣₜₐₗ | Insulin in portal vein | pmol/kg |
| I₁ | | pmol/L |
| I_{d} | Delayed insulin signal | pmol/L |
| Q_{solid} | Glucose in stomach at solid phase | mg |
| Q_{liquid} | Glucose in stomach at liquid phase | mg |
| Q_{gut} | Glucose in intestine | mg |
| I_{poly} | Non-monomeric insulin | pmol/kg |
| | in subcutaneous space | |
| Iₘₒₙₒ | Monomeric insulin in | pmol/kg |
| | subcutaneous space | |
| GL | Glucagon hormone | pg/ml |
| GLY | Pro-glycogen | mg |
| A_{GL} | Glucagon rate affected | [ ] |
| | endogenous glucose factor | |

GL accounts for diabetic I complications while satisfying normal patients in the glucagon system. GL is used in A_{GL}. GLY has two separate paths to the liver. With the exception of Y and A_{GL}, which are rates instead of physical quantities, all state variables must be non-negative. All basal quantities are marked by a subscript ‘b’ and are not time dependent.

Table 4, below, provides model parameters for the normal and the type 2 diabetic subject. Table 5, below, provides parameters of subcutaneous insulin kinetics, glucose sensor delay and PID controller. Table 6, below, provides additional constants.

TABLE 4

| | | | Type 2 | |
|---|---|---|---|---|
| | Param- | Normal | Diabetic | |
| Process | eter | Value | Value | Unit |
| Glucose | V_{G} | 1.88 | 1.49 | dl/kg |
| Kinetics | k₁ | 0.065 | 0.042 | min⁻¹ |
| | k₂ | 0.079 | 0.071 | min⁻¹ |
| Insulin | Vᵢ | 0.05 | 0.04 | l/kg |
| Kinetics | m₁ | 0.190 | 0.379 | min⁻¹ |
| | m₂ | 0.484 | 0.673 | min⁻¹ |
| | m₄ | 0.194 | 0.269 | min⁻¹ |
| | m₅ | 0.0364 | 0.0526 | min kg/pmol |
| | m₆ | 0.6471 | 0.8118 | dimensionless |
| | HE | 0.6 | 0.6 | dimensionless |
| Rate of | kₘₐₓ | 0.0558 | 0.0465 | min⁻¹ |
| Appearance | kₘᵢₙ | 0.0080 | 0.0076 | min⁻¹ |
| | K_{ab1} | 0.057 | 0.023 | min⁻¹ |
| | k | 0.558 | 0.0465 | min⁻¹ |
| | ƒ | 0.90 | 0.90 | dimensionless |
| | α | 0.00013 | 0.00006 | mg⁻¹ |
| | b | 0.82 | 0.68 | dimensionless |
| | c | 0.00236 | 0.00023 | mg⁻¹ |
| | d | 0.010 | 0.09 | dimensionless |
| Endogenous | k_{g1} | 2.70 | 3.09 | mg/kg/min |
| Production | k_{g2} | 0.0021 | 0.0007 | min⁻¹ |
| | k_{g3} | 0.009 | 0.005 | mg/kg/min per |
| | | | | pmol/l |
| | k_{g1} | 0.0618 | 0.0786 | mg/kg/min per |
| | | | | pmol/kg |
| | k₁ | 0.0079 | 0.0066 | min⁻¹ |
| Utilization | F | 1 | 1 | mg/kg/min |
| | V | 2.50 | 4.65 | mg/kg/min |
| | V | 0.047 | 0.034 | mg/kg/min |
| | | | | per pmol/l |
| | K | 225.59 | 466.21 | mg/kg |
| | P | 0.0331 | 0.0840 | min⁻¹ |
| Secretion | K | 2.30 | 0.99 | pmol/kg per |
| | | | | (mg/dl) |
| | α | 0.050 | 0.013 | min⁻¹ |
| | β | 0.11 | 0.05 | pmol/kg/min |
| | | | | per (mg/dl) |
| | γ | 0.5 | 0.5 | min⁻¹ |
| Renal | k_{c1} | 0.0005 | 0.0007 | min⁻¹ |
| Excretion | k_{c2} | 339 | 269 | mg/kg |

TABLE 5

| Control | Parameter | Value | Unit |
|---|---|---|---|
| Subcutaneous | kₐ | 0.0164 | min⁻¹ |
| insulin kinetics | kₐ₁ | 0.0018 | min⁻¹ |
| | kₐ₂ | 0.0182 | min⁻¹ |
| Glucose sensor | Tₐ | 10 | pmol/kg/min |
| delay | | | per mg/dl |
| PID controller | Kₚ | 0.032 | min |
| | T_{Q} | 66 | min |
| | Tₗ | 450 | min |

TABLE 6

ƒ_{GLY} = 0.25
GLY_{synth} = [0.2025927654630183 0.1865908261637011
0.0370319118341182 − 164.7045696728318700]
k₁_{_{A}} = 1/25
I_{A} = [1.21 − 1.14 1.66 − 0.88]
G_{M} = [1.425 − 1.406 0.619 − 0.49]
K_{GL}_{_{A}} = 1/65
G_{L}_{_{M}} = [0.7 0.37 − 36]
ƒ_{GNG resting} = 0.25
GLYₘₐₓ = 90000
t_{delay} = 3
GLY_{soft} = [0 1 − 1/1000 − GLYₘₐₓ]

FIG. 11 is a blood glucose 1101 versus time 1102 graph 1100 comparing glucose model predictions 1120 (line) to invasive blood glucose measurements 1110 (dots). The graph also illustrates the impact of insulin injections 1130 and meal intakes 1140.

A glucose estimator has been disclosed in detail in connection with various embodiments. These embodiments are disclosed by way of examples only and are not to limit the scope of the claims herein. One of ordinary skill in art will appreciate many variations and modifications.

1.-20. (canceled)

21. A system for blood glucose sensing, the system comprising:
one or more hardware signal processors configured to:
receive, from a noninvasive blood glucose sensor and an invasive blood glucose sensor, blood glucose data of a user;
receive user-specific data comprising food intake data and insulin intake data;
determine, using a glucose insulin estimator, a plurality of modeled blood glucose values over time between measurements by the blood glucose sensor, wherein the plurality of modeled blood glucose values are determined based on the blood glucose data and the user-specific data, wherein the glucose insulin estimator comprises state variables associated with at least one of insulin secretion rate, plasma glucose, tissue glucose, insulin in interstitial fluid, insulin in liver, insulin in plasma, insulin in portal veins, delayed insulin signal, glucose in the stomach at solid phase, glucose in the stomach at liquid phase, glucose in the intestine, non-monomeric insulin in the subcutaneous space, monomeric insulin in the subcutaneous space, glucagon hormone, pro-glycogen, or glucagon rate affected endogenous factor; and by dynamically optimizing a nonlinear state-space model of glucose and insulin reactions within a human body by optimizing parameters of the nonlinear state space model to minimize an error between modeled blood glucose values and measured values of blood glucose,
wherein an input of the state-space model comprises blood glucose data of the user;
determine a near-continuous estimate of blood glucose over a period of monitoring time based on a combination of the plurality of blood glucose values and the plurality of modeled blood glucose values; and
output a glucose trend over time to a display, the glucose trend based at least in part on the plurality of modeled blood glucose values.

22. The system of claim 21, wherein the glucose estimator comprises parameters associated with at least one of glucose kinetics, insulin kinetics, rate of appearance of insulin, endogenous production of insulin, utilization of insulin, secretion of insulin, or renal excretion of insulin.

23. The system of claim 21, wherein the noninvasive blood glucose sensor comprises an optical glucose sensor.

24. The system of claim 21, wherein the user-specific data further comprises one or more of biographical data and basal values.

25. The system of claim 21, wherein user-specific data is manually inputted.

26. The system of claim 21, the blood glucose estimator comprising a plurality of physiological sensors configured to provide sensor data associated with the user, the plurality of physiological sensors comprising an invasive sensor and a non-invasive sensor.

27. The system of claim 26,
wherein the one or more hardware signal processors are configured to generate a blood glucose estimate based at least in part on each of the plurality of modeled blood glucose values, a plurality of noninvasive sensor data and a plurality of invasive sensor data, and
wherein the one or more hardware signal processors are configured to recursively adjust parameters of a state-space model to minimize an error between the plurality of modeled blood glucose values of the user and measured values of blood glucose, the blood glucose estimate based at least in part on the state-space model having parameters resulting in minimal error between the plurality of modeled blood glucose values of the user and measured values of blood glucose.

28. The system of claim 27, wherein the state-space model comprises:
an input vector comprising an insulin intake and food intake;
a state vector comprising the state variables;
a state equation comprising the parameters; and
the modeled blood glucose values.

29. The system of claim 28, wherein the state equation comprises state variables associated with insulin secretion rate, plasma glucose, tissue glucose, insulin in interstitial fluid, insulin in liver, insulin in plasma, insulin in portal vein, delayed insulin signal, glucose in stomach at solid phase, glucose in stomach at liquid phase, glucose in intestine, non-monomeric insulin in subcutaneous space, monomeric insulin in subcutaneous space, glucagon hormone, pro-glycogen, and glucagon rate affected endogenous factor.

30. The system of claim 21, wherein the one or more hardware signal processors is configured to detect physiological events to generate a plurality of physiological event data based at least in part on the blood glucose data and independent of user input.

31. The system of claim 30, wherein an input of the state-space model comprises the plurality of physiological event data of the user.

32. A method for blood glucose monitoring, the method comprising:
receiving, using one or more hardware processors, blood glucose data of a user, the blood glucose data comprising measurements made by an invasive blood glucose sensor and a noninvasive blood glucose sensor;
receiving, using the one or more hardware processors, a plurality of user-specific data, the user-specific data comprising food intake data and insulin intake data;
determining, using the one or more hardware processors implementing a glucose insulin model, a plurality of modeled blood glucose values over time between measurements by the invasive blood glucose sensor, wherein the plurality of modeled blood glucose values are determined based on the blood glucose data and user-specific data, wherein the glucose insulin model is a nonlinear state-space model of glucose and insulin reactions within a human body, and wherein the glucose insulin model comprises:
state variables associated with at least one of insulin secretion rate, plasma glucose, tissue glucose, insulin in interstitial fluid, insulin in liver, insulin in plasma, insulin in portal vein, delayed insulin signal, glucose in stomach at solid phase, glucose in stomach at liquid phase, glucose in intestine, non-monomeric insulin in subcutaneous space, monomeric insulin in subcutaneous space, glucagon hormone, pro-glycogen, or glucagon rate affected endogenous factor;
dynamically optimizing the glucose insulin model to minimize an error between the plurality of modeled blood glucose values of the user and measured values of blood glucose based on the blood glucose data of the user; and
outputting a glucose trend over time to a display, the glucose trend based at least in part on the plurality of modeled blood glucose values.

33. The method of claim 32, wherein the glucose insulin model comprises parameters associated with at least one of glucose kinetics, insulin kinetics, rate of appearance of insulin, endogenous production of insulin, utilization of insulin, secretion of insulin, or renal excretion of insulin.

34. The method of claim 32, wherein the noninvasive blood glucose sensor is an optical sensor.

35. The method of claim 32, comprising detecting physiological events to generate a plurality of physiological event data based at least in part on the sensor data and independent of user input.
